(19) United States
(12) Patent Application Publication
(10) Pub. No.: US 2009/0092595 A1
(43) Pub. Date: Apr. 9, 2009

(54) Suppression of sPLA2-integrin binding for treating an inflammatory condition

(75) Inventors: Yoshikazu Takada, Davis, CA (US); Jun Saegusa, Davis, CA (US); Nobuaki Akakura, Hakodate (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 12/218,506

(22) Filed: Jul. 14, 2008

Related U.S. Application Data
(60) Provisional application No. 60/949,700, filed on Jul. 13, 2007.

(51) Int. Cl. A61K 39/395; G01N 33/567; A61K 31/7088

(52) U.S. Cl. 424/130.1; 435/7.2; 514/44

(57) ABSTRACT

The present invention relates to the discovery that a secretory phospholipase A2 (sPLA2-IIA) plays an active role in mediating inflammatory signaling by way of its specific binding with integrin, especially integrin αvβ3 or α4β1. More specifically, the invention provides a method for identifying inhibitors of inflammatory signaling by screening for compounds that interrupt the specific binding of sPLA2 and integrin. The invention also provides the novel use of a substance that suppresses the specific binding between sPLA2 and integrin for treating or preventing an inflammatory condition.

### CROSS-REFERENCES TO RELATED APPLICATIONS

This application claims priority to U.S. Provisional Patent Application No. 60/949,700, filed Jul. 13, 2007, the disclosure of which is incorporated by reference in its entirety.

### STATEMENT AS TO RIGHTS TO INVENTIONS MADE UNDER FEDERALLY SPONSORED RESEARCH OR DEVELOPMENT

This invention was made with support under Grant Nos. AG027350 and GM047157 by the National Institutes of Health. The government has certain rights in this invention.

### BACKGROUND OF THE INVENTION

Secretory phospholipase A2 group IIA (sPLA2-IIA) plays an important role in the pathogenesis of inflammatory diseases. Catalytic activity of this enzyme that generates arachidonic acid is a major target for development of anti-inflammatory agents. Independent of its catalytic activity, sPLA2-IIA induces pro-inflammatory signals in a receptor-mediated mechanism (e.g., through the M-type receptor). However, the M-type receptor is species-specific: sPLA2-IIA binds to the M-type receptor in rodents and rabbits, but not in human. Thus sPLA2-IIA receptors in human have not been established. The present inventors have demonstrated that sPLA2-IIA bound to integrin αvβ3 at a high affinity (KD=2×10⁻⁷M). They identified amino acid residues in sPLA2-IIA (Arg-74 and Arg-100) that are critical for integrin binding using docking simulation and mutagenesis. The integrin-binding site did not include the catalytic center or the M-type receptor-binding site. sPLA2-IIA also bound to α4β1. The inventors showed that sPLA2-IIA competed with VCAM-1 for binding to α4β1, and bound to a site close to those for VCAM-1 and CS-1 in the α4 subunit. Wt and the catalytically inactive H47Q, mutant of sPLA2-IIA-induced cell proliferation and ERK1/2 activation in monocytic cells, but the integrin-binding-defective R74E/R100E mutant did not. This indicates that integrin binding is required, but catalytic activity is not required, for sPLA2-IIA-induced proliferative signaling. These results indicate that integrins αvβ3 and α4β1 serve as receptors for sPLA2-IIA and mediate pro-inflammatory action of sPLA2-IIA, and that integrin-sPLA2-IIA interaction is a novel therapeutic target to suppress pro-inflammatory responses and therefore treat diseases or conditions involving inflammation.

### BRIEF SUMMARY OF THE INVENTION

In one aspect, the present invention provides a method for identifying an inhibitor for integrin-sPLA2-IIA binding. The method comprises the steps of: (a) contacting a test compound with sPLA2-IIA and integrin αvβ3, or with sPLA2-IIA and integrin α4β1, under conditions that permit specific binding between sPLA2-IIA and integrin αvβ3 or integrin α4β1; and (b) determining the level of specific binding between sPLA2-IIA and integrin αvβ3 or integrin α4β1, wherein a decrease in the level of specific binding compared to a control level of specific binding between sPLA2-IIA and integrin αvβ3 or integrin α4β1 under the same conditions but in the absence of the test compound indicates the compound as an inhibitor for integrin-sPLA2-IIA binding. In some embodiments, integrin αvβ3 or integrin α4β1 is present on the surface of a cell. Integrin αvβ3 or integrin α4β1 may be endogenously expressed by the cells on their surface or recombinantly expressed on the cell surface. In other embodiments, sPLA2-IIA, or integrin αvβ3, or integrin α4β1 is immobilized on a solid support. In some cases, sPLA2-IIA is labeled with a fluorescent dye, such as fluorescein isothiocyanate (FITC).

In the cases where the method is performed using cells expressing integrin αvβ3 or integrin α4β1 on the cell surface, the level of specific binding between sPLA2-IIA and integrin can be detected directly, or can be determined indirectly by measuring the level of activation of at least one MAP kinase, such as ERK1 or ERK2. In the alternative, the level of specific binding between sPLA2-IIA and integrin can be determined indirectly by measuring the level of proliferation of the cells expressing the integrin αvβ3 or integrin α4β1 proteins, for example, the U937 human monocytic lymphoma cells or the K562 cells.

In a second aspect, the invention provides a method for treating or preventing an inflammatory condition, comprising the step of administering to a subject an effective amount of an inhibitor for sPLA2-IIA and integrin αvβ3 binding or sPLA2-IIA and integrin α4β1 binding. In some embodiments, the inhibitor is an inactivating antibody of sPLA2-IIA or integrin αv, α4, β1, or β3. In other embodiments, the inhibitor is an inhibitory nucleic acid comprising a sequence complementary to an sPLA2-IIA or integrin αv, α4, β1, or β3 polynucleotide.

In a third aspect, the present invention provides a composition comprising (1) an effective amount of an inhibitor for sPLA2-IIA and integrin αvβ3 binding or sPLA2-IIA and integrin α4β1 binding and (2) a pharmaceutically acceptable carrier. In some embodiments, the inhibitor is an inactivating antibody of sPLA2-IIA or integrin αv, α4, β1, or β3. In other embodiments, the inhibitor is an inhibitory nucleic acid comprising a sequence that is complementary to an sPLA2-IIA or integrin αv, α4, β1, or β3 polynucleotide. Optionally, the composition may further comprise an additional therapeutic compound.

In a fourth aspect, the present invention provides a kit for treating an inflammatory condition, said kit comprising the composition that comprises (1) an effective amount of an inhibitor for sPLA2-IIA and integrin αvβ3 binding or sPLA2-IIA and integrin α4β1 binding and (2) a pharmaceutically acceptable carrier. In some embodiments, the inhibitor is an inactivating antibody of sPLA2-IIA or integrin αv, α4, β1, or β3. In other embodiments, the inhibitor is an inhibitory nucleic acid comprising a sequence that is complementary to an sPLA2-IIA or integrin αv, α4, β1, or β3 polynucleotide. Optionally, the composition may further comprise an additional therapeutic compound.

### BRIEF DESCRIPTION OF THE DRAWINGS

FIG. 1. sPLA2-IIA binding to integrin αvβ3. a) Cell adhesion to sPLA2-IIA in an αvβ3- and proteoglycans-dependent manner. Wells of a 96-well microtiter plate were coated with sPLA2-IIA at indicated coating concentrations. Remaining protein-binding sites were blocked with BSA. CHO cells expressing recombinant αvβ3 (β3-CHO), mock-transfected CHO cells, proteoglycans-deficient CHO cell variant (pgs745), expressing recombinant αvβ3 (β3-745), and mock-transfected pgs745 cells (10⁵ cells per well in 100 μl Tyrodes-HEPES with 1 mM MgCl₂) were added to the wells. After incubating for 1 h at 37° C., unbound cells were removed by gentle rinsing and bound cells were quantified using endogenous phosphatase activity (Prater, C. A. et al., J. Cell Biol., 112:1031-1040 (1991)). Data is shown as means+/−SEM of triplicate experiments. b) Effect of anti-(33 mAb 7E3 on adhesion of β3-CHO cells to immobilized sPLA2-IIA. Adhesion assays were done as described in FIG. 1a. mAb 7E3 (specific to human β3 subunit, function blocking) or purified mouse IgG as a negative control was added to the medium during adhesion assays at 10 μg/ml. *P<0.05 between control IgG and anti-03 (7E3) by t-test. c) Binding of FITC-labeled sPLA2-IIA to αvβ3 on the cell surface. β3-CHO cells (about 50% are positive in αvβ3 expression) were harvested with 3.5 mM EDTA in PBS. Cells were double-stained with i) FITC-labeled sPLA2-IIA (10 μg/ml in the presence of 10 mM Mg²⁺ at room temperature for 30 min), and ii) with non-blocking anti-human integrin β3 subunit mAb AV10 and PE (phycoerythrin) conjugated secondary antibody. Bound FITC (FL1) and PE (FL2) were quantified in flow cytometry. FITC binding to the PE-positive population (αvβ3-high) and the PE-negative population (αvβ3-low) is shown. d) Binding of recombinant soluble αvβ3 to immobilized sPLA2-IIA. Soluble αvβ3 (with a 6His tag at the C-terminus of the β3 subunit) was incubated with sPLA2-IIA, the ADAM15 disintegrin domain (a positive control) (Zhang, X. P. et al., J. Biol. Chem., 273(13):7345-7350 (1998)), and BSA, which were immobilized to wells of a 96-well-microtiter plate (20 μg/ml coating concentration). Remaining protein-binding sites were blocked with BSA. Bound αvβ3 was detected using peroxidase-conjugated anti-6His antibody. Bound peroxidase activity was measured. Data is shown as means+/−SEM of triplicate experiments. *P<0.05 between sPLA2-IIA and BSA by t-test.

FIG. 2. Docking simulation of αvβ3-sPLA-IIA interaction. a) A model of αvβ3-sPLA2-IIA interaction from cluster 1, in which 24 of the 50 docking poses clustered with the lowest docking energy (−25.5 Kcal/mol) within 0.5 angstrom RMSD. b) Several amino acid residues within the predicted integrin-binding interface of sPLA2-IIA.

FIG. 3. Localization of the integrin-binding site of sPLA2-IIA. a) Binding of sPLA2-IIA mutants to soluble αvβ3. Based on the docking model, we introduced mutations in the integrin-binding interface of sPLA2-IIA. The mutant proteins were tested for binding to soluble integrin αvβ3. The low level background binding to BSA was subtracted. b) Summary of the mutagenesis study of sPLA2-IIA-integrin interaction. The binding of soluble αvβ3 to immobilized wt and mutant sPLA2-IIA was determined at the saturating conditions (20 μg/ml coating concentrations). The H47Q mutation is located in the catalytic center of the enzyme. c) The surface plasmon resonance (SPR) study of sPLA2-IIA binding to αvβ3. Soluble integrin αvβ3 was immobilized to a sensor chip and the binding of wt sPLA2-IIA, and the H47Q and R74E/R100E mutants (Concentrations at 2, 1, and 0.5 nM) was analyzed. KD was calculated as 2.11×10⁻⁷M for wt sPLA2-IIA, 4.47×10⁻⁸M for H47Q, and 1.08×10⁻⁶ M for R74E/R100E. d) Effect of sPLA2-IIA mutations on PLA2 activity. PLA2 activity was measured as described in the Examples section. A similar result was obtained from another independent experiment.

FIG. 4. sPLA2-IIA-induced proliferation of U937 human monocytic lymphoma cells in an integrin-dependent manner. a) Effect of sPLA2-IIA mutants on cell proliferation. U937 cells (αvβ3+, α4β1+) were plated in wells of 96-well plates (10,000 cells/well), and serum-starved for 48 h. After treatment with wt or mutant sPLA2-IIA for 48 h, we measured cell proliferation by MTS assays. P<0.0001 between wt and R74E/R100E by 2-way ANOVA. b) wt and catalytically inactive H47Q mutant of sPLA2-IIA induced ERK1/2, but integrin-binding-defective R74E/R100E mutant did not. U937 cells were serum-starved for 24 h, and stimulated with wt and mutant sPLA2-IIA (0.5 μg/ml) for 10 min at 37° C. Cell lysates were analyzed by western blotting with anti-phospho ERK1/2 or anti-ERK1/2 antibodies. The blot is representative of three independent experiments.

FIG. 5. sPLA2-IIA binding to integrin α4β1. a) Cells expressing recombinant α4β1 adhered to wt sPLA2-IIA better than cells expressing other recombinant integrins. We used transfected K562 cells clonally express different human integrins for adhesion assays. Low background adhesion to BSA was subtracted. Data is shown as means+/−SEM of triplicate experiments. b) sPLA2-IIA blocked adhesion of U937 cells to VCAM-1, but did not block adhesion of U937 cells to the cell-binding domain of fibronectin. Wells of 96-well microtiter plate were coated with VCAM-1 and FN-GST, and incubated with U937 cells (1×10⁴ cells/plate) in the presence of the increasing concentrations of sPLA2-IIA for 1 h at 37° C. Adherent cells were quantified using endogenous phosphatase after gently rinsing the well to remove unbound cells. Data is shown as means+/−SEM of triplicate experiments. *P=0.0101 and **P<0.0001 by t-test. c) Amino acid sequence in the α4 subunit that is critical for VCAM-1 and CS-1 binding is also critical for sPLA2-IIA binding. CHO cells that clonally express wt or mutant α4 (Irie, A. et al., Embo J, 14(22): 5550-5556 (1995)) were used for adhesion assays. Data is shown as means+/−SEM of triplicate experiments. Similar results were obtained using K562 cells expressing α4 mutants (not shown).

FIG. 6. sPLA2-induced proliferation of K562 cells in an integrin-dependent and catalytic activity-independent manner. a) wt sPLA2-IIA enhanced proliferation of K562 cells that clonally express αvβ3 or α4β1, but not mock-K562 cells. Cells were serum-starved for 48 h and stimulated with sPLA2-IIA for 48 h. Cell proliferation was determined by MTS assays. Data is shown as means+/−SEM of triplicate experiments. α4- and αvβ3-K562 cells proliferated faster than mock-K562 cells. P<0.0001 (α4-K562) and **P=0.0353 (αvβ3-K562) compared to mock K562 cells by 2-way ANOVA. b) sPLA2-IIA-induced cell proliferation required integrin binding but did not require catalytic activity. Serum-starved cells were stimulated with wt or mutant sPLA2-IIA (0.5 μg/ml) for 48 h. Data is shown as means+/−SEM of triplicate experiments. α4-K562 and αvβ3-K562 cells grew faster than mock K562 cells with wt and H47Q sPLA2-IIA (*P<0.05 by t-test).

### DEFINITIONS

“Inflammation” or an “inflammatory response” refers to an organism's immune response to irritation, toxic substances, pathogens, or other stimuli. The response can involve innate immune components and/or adaptive immunity. Inflammation is generally characterized as either chronic or acute. Acute inflammation is characterized by redness, pain, heat, swelling, and/or loss of function due to infiltration of plasma proteins and leukocytes to the affected area. Chronic inflammation is characterized by persistent inflammation, tissue destruction, and attempts at repair. Monocytes, macrophages, plasma B cells, and other lymphocytes are recruited to the affected area, and angiogenesis and fibrosis occur, often leading to scar tissue.

An “inflammatory condition” is one characterized by an inflammatory response, as described above. A list of exemplary inflammatory conditions includes: asthma, autoimmune disease, chronic inflammation, chronic prostatitis, glomerulonephritis, hypersensitivities and allergies, skin disorders such as eczema, inflammatory bowel disease, pelvic inflammatory disease, reperfusion injury, rheumatoid arthritis, transplant rejection, and vasculitis.

As used herein, “sPLA2-IIA” refers to a member of the phospholipase A2 (PLA2) family, a secreted phospholipase. In this application, an “sPLA2-IIA protein” refers to a full-length sPLA2-IIA polypeptide sequence, including the human sPLA2-IIA (GenBank Accession No. P14555, encoded by GenBank Accession No. M22430), its polymorphic variants and species orthologs or homologs. An “sPLA-IIA polynucleotide” refers to a nucleic acid sequence from the gene encoding the sPLA2-IIA protein and may include both the coding and non-coding regions. “sPLA2-IIA cDNA,” “sPLA2-IIA mRNA,” “sPLA2-IIA coding sequence,” and their variations refer to a nucleic acid sequence that encodes an sPLA2-IIA polypeptide.

Similarly, integrin chains αv, α4, β1, and β3 are exemplified by human integrin αv, α4, β1, and β3 (GenBank Accession Nos. P06756, P13612, P05556, and P05106, encoded by GenBank Accession Nos. M14648, X16983, X07979, and J02703, respectively). Each of these terms encompasses its corresponding polymorphic variants and interspecies orthologs/homologs. “Integrin αvβ3” refers to a heterodimer of integrin αv and β3 chains, and “integrin α4β3” refers to a heterodimer of integrin αv and β1 chains.

“Inhibitors” or “suppressors” of sPLA2-IIA and integrin binding refer to compounds that have an inhibitory or disruptive effect on the specific binding between sPLA2-IIA and integrin αvβ3 or α4β1, as identified in in vitro and in vivo binding assays described herein. In some cases, an inhibitor directly binds to either sPLA2-IIA or integrin chain αv, β3, α4, or β1, such that specific binding between sPLA2-IIA and integrin αvβ3 or α4β1 is suppressed or abolished. For instance, an antibody that specifically binds either sPLA2-IIA or integrin chain αv, β3, α4, or β1. Inhibitors also include compounds that are capable of reducing the expression of sPLA2-IIA or integrin chains αvβ3 or α4β1 at the protein level, e.g., transcription-based inhibitors, such as antisense RNAs and siRNAs, RNA aptamers, and the like. Assays for inhibitors of sPLA2-IIA-integrin binding include, e.g., applying putative inhibitor compounds to a cell expressing the appropriate integrin(s) in the presence of sPLA2-IIA under conditions that permit sPLA2-IIA-integrin binding and then determining the effect of the compounds on the binding, as described herein. Assays for the inhibitors also include cell-free systems, where samples comprising sPLA2-IIA and the appropriate integrin(s) treated with a candidate inhibitor are compared to a control sample without the inhibitor to examine the extent of inhibition on the sPLA2-IIA-integrin binding. Control samples (not treated with inhibitors) are assigned a relative binding level of 100%. Inhibition of binding is achieved when the level of binding or downstream signal transduction relative to the control is about 90%, 80%, 70%, 50%, 20%, 10% or close to 0%.

A composition “consisting essentially of a sPLA2-IIA-integrin binding inhibitor” is one that includes an inhibitor of specific binding between sPLA2-IIA and integrin αvβ3 or α4β1, but no other compounds that contribute significantly to the inhibition of the binding. Such compounds may include inactive excipients, e.g., for formulation or stability of a pharmaceutical composition, or active ingredients that do not significantly contribute to the inhibition of sPLA2-integrin binding. Exemplary compounds consisting essentially of a sPLA2-integrin inhibitor include therapeutics, medicaments, and pharmaceutical compositions.

An “inactivating antibody” is an antibody or antibody fragment (e.g., an Fab fragment) that binds specifically to a target molecule, such as sPLA2-IIA or integrin αvβ3 or α4β1 and interferes with, reduces, inhibits, or completely block the signal transduction resulted from sPLA2-IIA-integrin binding, as compared to the signal transduction of the same nature in the absence of such inactivating antibody.

As used herein, an “effective amount” or a “therapeutically effective amount” means the amount of a compound that, when administered to a subject or patient for treating a disorder, is sufficient to prevent, reduce the frequency of, or alleviate the symptoms of the disorder. The effective amount will vary depending on a variety of the factors, such as a particular compound used, the disease and its severity, the age, weight, and other factors of the subject to be treated. Amelioration of a symptom of a particular condition by administration of a pharmaceutical composition described herein refers to any lessening, whether permanent or temporary, that can be associated with the administration of the pharmaceutical composition. For example, the amount of an inhibitor of sPLA2-IIA-integrin binding is considered therapeutically effective for treating an inflammatory condition when treatment results in eliminated symptoms, delayed onset of symptoms, or reduced frequency or severity of symptoms such as discomfort, irritation, swelling, etc.

A “subject,” or “subject in need of treatment,” as used herein, refers to an individual who seeks medical attention due to risk of, or actual suffering from, a condition involving an undesirable inflammatory reaction. The term subject can include both animals and humans. Subjects or individuals in need of treatment include those that demonstrate symptoms of the inflammatory condition or are at risk of suffering from these symptoms.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The term “target nucleic acid” refers to a nucleic acid (often derived from a biological sample) to which a nucleic acid probe or inhibitory nucleic acid is designed to specifically hybridize. The target nucleic acid has a sequence that is complementary to the nucleic acid sequence of the corresponding probe directed to the target. The term target nucleic acid may refer to the specific subsequence of a larger nucleic acid to which the inhibitory nucleic acid or probe is directed or to the overall sequence (e.g., gene or mRNA) whose expression level it is desired to target. The difference in usage will be apparent from context. For example, a sPLA2-IIA target sequence can comprise a portion of the coding sequence, a portion of non-coding sequence, or the entire sPLA2-IIA gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see Paul, Fundamental Immunology, Third Ed., Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or those synthesized de novo using recombinant DNA methodologies (e.g., single chain Fv). These antibody fragments are also useful for methods requiring antigen recognition.

Chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient.

“Humanized” antibodies combine an even smaller portion of the non-human antibody with human components. Generally, a humanized antibody comprises the hypervariable regions, or complementarily determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see, e.g., Jones et al., Nature, 321:522-525 (1986)).

The phrase “specifically (or selectively) binds to an antibody” or “specifically (or selectively) immunoreactive with,” when referring to a protein or peptide, refers to a binding reaction that is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein, e.g., sPLA2-IIA or integrin chain αv, β3, α4, or β1. For example, antibodies raised against sPLA2-IIA can be selected to obtain antibodies specifically immunoreactive with that protein and not with other proteins, except for polymorphic variants, e.g., proteins at least 80%, 85%, 90%, 95%, or 99% identical to sPLA2-IIA or a fragment thereof, e.g., a domain or unique subsequence. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

### DETAILED DESCRIPTION OF THE INVENTION

### I. Introduction

The phospholipase A2 (PLA2) family is a group of intracellular and secreted enzymes that hydrolyzes the sn-2 ester bond in the glyceroacyl phospholipids present in lipoproteins and cell membranes to form nonesterified fatty acids and lysophospholipids. These products act as intracellular second messengers or are further metabolized into potent mediators of a broad range of cellular processes, including inflammation, apoptosis, and atherogenesis (Tatulian, S. A., Biophys J, 80(2):789-800 (2001)). The mammalian secretory PLA2 isoforms are comprised of the groups named IB, IIA, IIC, IID, IIE, IIF, V, X, and XII (Six, D. A. and Dennis, E. A., Biochim Biophys Acta, 1488(1-2):1-19 (2000); Gelb, M. H. et al., J Biol Chem, 275(51):39823-39826 (2000)). All secretory PLA2 isoforms have in common a Ca²⁺-dependent catalytic mechanism, a low molecular weight (13 to 16 kDa), several disulfide bridges, and a well-conserved overall 3D structure (Six, D. A. and Dennis, E. A., Biochim Biophys Acta, 1488(1-2):1-19 (2000); Gelb, M. H. et al., Curr Opin Struct Biol, 9(4):428-432 (1999); Valentin, E. and Lambeau, G., Biochim Biophys Acta, 1488(1-2):59-70 (2000)). Secretory PLA2 type IIA (sPLA2-IIA) was first isolated and purified from rheumatoid synovial fluid. sPLA2-IIA is an acute-phase reactant and is found in markedly increased plasma concentrations in diseases that involve systemic inflammation such as sepsis, rheumatoid arthritis, and cardiovascular disease (up to 1000-fold and >1 μg/ml). Inflammatory cytokines such as IL-6, TNF-α, and IL-1β induce synthesis and release of sPLA2-IIA in arterial smooth muscle cells and hepatocytes, which are the major sources of the plasma sPLA2-IIA in these systemic inflammatory conditions (Jaross, W. et al., Eur J Clin Invest, 32(6):383-393 (2002); Niessen, H. W. et al., Cardiovasc Res, 60(1):68-77 (2003)). In addition to being a pro-inflammatory protein, sPLA2-IIA expression is elevated in neoplastic prostatic tissue (Jiang, J. et al., Am J Pathol, 160(2):667-671 (2002)) and dysregulation of sPLA2-IIA may play a role in prostatic carcinogenesis (Dong, Q. et al., Cancer Lett, 240(1):9-16 (2006)), and is a potential therapeutic target in prostate cancer (Sved, P. et al., Cancer Res, 64(19):6934-6940 (2004)).

Notably some biological effects associated with sPLA2-IIA are independent of its catalytic function (Tada, K. et al., J Immunol, 161(9):5008-5015 (1998)). Catalytically inactive sPLA2-IIA mutants retained the ability to enhance cyclooxygenase-2 expression in connective tissue mast cells (Tada, K. et al., J Immunol, 161(9):5008-5015 (1998)). Also inactivation of sPLA2-IIA by bromophenacyl bromide did not affect sPLA2-IIA's ability to induce secretion of β-glucuronidase, IL-6, and IL-8 from human eosinophils (Triggiani, M. et al., J Immunol, 170(6):3279-3288 (2003)). It has thus been proposed that sPLA2-IIA's action is mediated through interaction with specific receptors. Indeed the enzyme binds to a high affinity receptor of 180 kDa present on rabbit skeletal muscle (Lambeau, G. et al., J Biol Chem, 269(3):1575-1578 (1994)). This so-called M (muscle) type receptor belongs to the superfamily of C-type lectins and mediates some of the physiological effects of mammalian sPLA2-IIA, and binding of sPLA2-IIA to this receptor induces internalization of sPLA2-IIA (Nicolas, J. P. et al., J Biol Chem, 270(48):28869-28873 (1995)). However, the interaction between sPLA2-IIA and the M-type receptor is species-specific, and human sPLA2-IIA binds to the human or mouse M-type receptor very weakly (Cupillard, L. et al., J Biol Chem, 274(11):7043-7051 (1999)). Thus, sPLA2-IIA receptors in human have not been established. Mammalian sPLA2-IIAs bind to heparan sulfate proteoglycans like glypican-1 (Murakami, M. et al., J Biol Chem, 274(42):29927-29936 (1999)) and decorin in apoptotic human T cells (Sartipy, P. et al., Circ Res, 86(6):707-714 (2000)). The binding of sPLA2-IIA to heparan sulfate proteoglycans has been implicated in the release of arachidonic acid from apoptotic T cells (Boilard, E. et al., Faseb J, 17(9):1068-1080 (2003), but it is unclear whether this process plays a role in other situations.

Integrins are a family of cell adhesion receptors that recognize ECM ligands and cell surface ligands (Hynes, R. O., Cell, 110(6):673-687 (2002)). Integrins are proteins of transmembrane αβ heterodimers, and at least 18 α and 8 β subunits are known (Shimaoka, M. and Springer, T. A., Nat Rev Drug Discov, 2(9):703-716 (2003)). Integrins transduce signals to the cell upon ligand binding (Hynes, R. O., Cell, 110(6):673-687 (2002)). In this study, we investigated whether integrins are involved in the pro-inflammatory functions of sPLA2-IIA. Here we demonstrate that sPLA2-IIA bound to integrins and induced proliferative signals in an integrin-dependent manner. We first showed that sPLA2-IIA specifically bound to integrin αvβ3 at a high affinity in several different assays, and localized the integrin-binding site in sPLA2-IIA using docking simulation and mutagenesis. The integrin-binding site did not include the catalytic center or the M-type receptor-binding site. We obtained evidence that sPLA2-IIA also bound to α4β1 and competed with vascular cell adhesion molecule-1 for binding to α4β1. Wt and the catalytically inactive mutant of sPLA2-IIA-induced cell proliferation, but an integrin-binding defective mutant did not induce cell proliferation in cells that express αvβ3 and/or α4β1. This indicates that integrin binding is required, but catalytic activity is not required, for sPLA2-IIA-induced cell proliferation. sPLA2-IIA-induced cell proliferation of monocytic U937 cells (αvβ3+/α4β1+) and induced ERK1/2 activation in an integrin-dependent manner. These results suggest that integrins αvβ3 and α4β1 may serve as receptors for sPLA2-IIA and mediate pro-inflammatory action of sPLA2-IIA in human. Thus integrin-sPLA2-IIA interaction is a novel therapeutic target in inflammation.

### II. Recombinant Expression of Polypeptides

A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The polynucleotide sequence encoding a polypeptide of interest, e.g., an sPLA2-IIA or integrin polypeptide, and synthetic oligonucleotides can be verified after cloning or subcloning using, e.g., the chain termination method for sequencing double-stranded templates of Wallace et al., Gene 16: 21-26 (1981).

B. Cloning and Subcloning of a Coding Sequence

The polynucleotide sequences encoding human sPLA2-IIA, integrin αv, α4, β1, and β3 are known as GenBank Accession No. M22430, M14648, X16983, X07979, and J02703, respectively. The corresponding amino acid sequences are P14555, P06756, P13612, P05556, and P05106, respectively. These polynucleotide sequences may be obtained from a commercial supplier or by amplification methods such as polymerase chain reaction (PCR).

The rapid progress in the studies of human genome has made possible a cloning approach where a human DNA sequence database can be searched for any gene segment that has a certain percentage of sequence homology to a known nucleotide sequence. Any DNA sequence so identified can be subsequently obtained by chemical synthesis and/or PCR technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a polynucleotide sequence encoding an sPLA2-IIA or integrin chain can be isolated from a cDNA or genomic DNA library using standard cloning techniques such as PCR, where homology-based primers can often be derived from a known nucleic acid sequence encoding an sPLA2-IIA or integrin polypeptide. This approach is particularly useful for identifying variants, orthologs, or homologs of sPLA2-IIA or integrin chains such as αv, α4, β1, and β3. Most commonly used techniques for this purpose are described in standard texts, e.g., Sambrook and Russell, supra.

cDNA libraries suitable for obtaining a coding sequence for a human sPLA2-IIA or integrin chain may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the gene of interest (e.g., sPLA2-IIA or integrin αv, α4, β1, or β3 chain) from the cDNA library. A general description of appropriate procedures can be found in Sambrook and Russell, supra. A similar procedure can be followed to obtain a sequence encoding a human sPLA2-IIA or integrin chain from a human genomic library, which may be commercially available or can be constructed according to various art-recognized methods. Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library.

Upon acquiring a polynucleotide sequence encoding an sPLA2-IIA or integrin chain, the sequence can then be subcloned into a vector, for instance, an expression vector, so that a recombinant polypeptide can be produced from the resulting construct. Further modifications to the coding sequence, e.g., nucleotide substitutions, may be subsequently made to alter the characteristics of the polypeptide.

C. Modification of a Polynucleotide Coding Sequence

The amino acid sequence of a human sPLA2-IIA or integrin chain may be modified while maintaining or enhancing the polypeptide's capability to inhibit endothelial cell proliferation, as determined by the in vitro or in vivo methods described below. Possible modifications to the amino acid sequence may include conservative substitutions; deletion or addition of one or more amino acid residues (e.g., addition at one terminal of the polypeptide of a tag sequence such as 6×His to facilitate purification or identification); truncation of a fragment ranging from approximately 1, 2, 3, 4, 5, 6, 7, 8, 9, 10, 15, 20, or up to 30 amino acids of the polypeptide at either or both of the N- and C-termini.

A variety of mutation-generating protocols are established and described in the art, and can be readily used to modify a polynucleotide sequence encoding an sPLA2-IIA or integrin polypeptide. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

D. Modification of Nucleic Acids for Preferred Codon Usage in a Host Organism

The polynucleotide sequence encoding an sPLA2-IIA or integrin polypeptide can be further altered to coincide with the preferred codon usage of a particular host. For example, the preferred codon usage of one strain of bacterial cells can be used to derive a polynucleotide that encodes an sPLA2-IIA polypeptide and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the sPLA2-IIA or integrin polypeptides.

E. Chemical Synthesis of Polypeptides

The amino acid sequences of human sPLA2-IIA, integrin αv, α4, β1, and β3 chains have been established (e.g., GenBank Accession Nos. P14555, P06756, P13612, P05556, and P05106). Polypeptides of known sequences, especially those of relatively short length such as human sPLA2-IIA, may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### III. Expression and Purification of Recombinant Polypeptides

Following verification of the coding sequence, a polypeptide of interest can be produced using routine techniques in the field of recombinant genetics, relying on the polynucleotide sequences encoding the polypeptide disclosed herein.

A. Expression Systems

To obtain high level expression of a nucleic acid encoding a polypeptide of interest, one typically subclones the polynucleotide coding sequence into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the sPLA2-IIA or integrin polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the desired polypeptide in host cells. A typical expression cassette thus contains a promoter operably linked to the nucleic acid sequence encoding the polypeptide and signals required for efficient polyadenylation of the transcript, ribosome binding sites, and translation termination. The nucleic acid sequence encoding the desired polypeptide is typically linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. If, however, a recombinant polypeptide (such as an integrin chain αv, α4, β1, or β3) is intended to be expressed on the host cell surface, an appropriate anchoring sequence is used in concert with the coding sequence. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the desired polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., an sPLA2-IIA or integrin chain) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that various conservative substitutions can be made to any sPLA2-IIA or integrin chains or its coding sequence while still retaining the biological activity of the polypeptide, e.g., the ability to transduce pro-inflammatory signals. Moreover, modifications of a polynucleotide coding sequence may also be made to accommodate preferred codon usage in a particular expression host without altering the resulting amino acid sequence.

B. Transfection Methods

Standard transfection methods are used to produce bacterial, mammalian, yeast, insect, or plant cell lines that express large quantities of a recombinant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

C. Purification of Recombinantly Produced Polypeptides

Once the expression of a recombinant polypeptide in transfected host cells is confirmed, e.g., by an immunological assay, the host cells are then cultured in an appropriate scale for the purpose of purifying the recombinant polypeptide.

1. Purification of Recombinantly Produced Polypeptide from Bacteria

When desired polypeptides are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

When a recombinant polypeptide is expressed in host cells in a soluble form, its purification can follow the standard protein purification procedure described below. This standard purification procedure is also suitable for purifying polypeptides obtained from chemical synthesis (e.g., a human sPLA2-IIA polypeptide).

i. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., an sPLA2-IIA or integrin monomer polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as an sPLA2-IIA or integrin chain) can also be separated from other proteins on the basis of their size, net surface charge, hydrophobicity, or affinity for ligands. In addition, antibodies raised against an sPLA2-IIA or an integrin chain (αv, β3, α4, or β1) can be conjugated to column matrices and the corresponding polypeptide immunopurified. All of these methods are well known in the art.

It will be apparent to one of skill that chromatographic techniques can be performed at any scale and using equipment from many different manufacturers (e.g., Pharmacia Biotech).

### IV. Inhibitors of sPLA2-IIA and Integrin Binding

A. Inhibitory Nucleic Acids

Inhibition of sPLA2-IIA or integrin αv, β3, α4, or β1 gene expression can be achieved through the use of inhibitory nucleic acids. Inhibitory nucleic acids can be single-stranded nucleic acids or oligonucleotides that can specifically bind to a complementary nucleic acid sequence. By binding to the appropriate target sequence, an RNA-RNA, a DNA-DNA, or RNA-DNA duplex or triplex is formed. These nucleic acids are often termed “antisense” because they are usually complementary to the sense or coding strand of the gene, although recently approaches for use of “sense” nucleic acids have also been developed. The term “inhibitory nucleic acids” as used herein, refers to both “sense” and “antisense” nucleic acids.

In one embodiment, the inhibitory nucleic acid can specifically bind to a target sPLA2-IIA or integrin αv, β3, α4, or β1 polynucleotide. Administration of such inhibitory nucleic acids can inhibit undesired inflammatory responses by reducing or eliminating the effects of sPLA2-IIA-intergrin binding and its downstream signals. Nucleotide sequences encoding sPLA2-IIA and integrin chains αv, β3, α4, and β1 are known for several species, including the human cDNA (Genbank Accession Numbers provided above). One can derive a suitable inhibitory nucleic acid from the human sPLA2-IIA or integrin αv, α4, β1, or β3, and their polymorphic variants or interspecies orthologs/homologs.

By binding to the target nucleic acid, the inhibitory nucleic acid can inhibit the function of the target nucleic acid. This could, for example, be a result of blocking DNA transcription, processing or poly(A) addition to mRNA, DNA replication, translation, or promoting inhibitory mechanisms of the cells, such as promoting RNA degradation. Inhibitory nucleic acid methods therefore encompass a number of different approaches to altering expression of specific genes that operate by different mechanisms. These different types of inhibitory nucleic acid technology are described in Helene and Toulme, Biochim. Biophys. Acta., 1049:99-125 (1990).

Inhibitory nucleic acid therapy approaches can be classified into those that target DNA sequences, those that target RNA sequences (including pre-mRNA and mRNA), those that target proteins (sense strand approaches), and those that cause cleavage or chemical modification of the target nucleic acids.

Approaches targeting DNA fall into several categories. Nucleic acids can be designed to bind to the major groove of the duplex DNA to form a triple helical or “triplex” structure. Alternatively, inhibitory nucleic acids are designed to bind to regions of single stranded DNA resulting from the opening of the duplex DNA during replication or transcription. See Helene and Toulme, supra.

More commonly, inhibitory nucleic acids are designed to bind to mRNA or mRNA precursors. Inhibitory nucleic acids are used to prevent maturation of pre-mRNA. Inhibitory nucleic acids may be designed to interfere with RNA processing, splicing or translation. The inhibitory nucleic acids are often targeted to mRNA. In this approach, the inhibitory nucleic acids are designed to specifically block translation of the encoded protein. Using this approach, the inhibitory nucleic acid can be used to selectively suppress certain cellular functions by inhibition of translation of mRNA encoding critical proteins. For example, an inhibitory antisense nucleic acid complementary to regions of a target mRNA inhibits protein expression (see, e.g., Wickstrom et al., Proc. Nat'l. Acad. Sci. USA, 85:1028-1032 (1988); and Harel-Bellan et al., Exp. Med., 168:2309-2318 (1988)). As described in Helene and Toulme, supra, inhibitory nucleic acids targeting mRNA have been shown to work by several different mechanisms in order to inhibit translation of the encoded protein(s).

The inhibitory nucleic acids introduced into the cell can also encompass the “sense” strand of the gene or mRNA to trap or compete for the enzymes or binding proteins involved in mRNA translation. See Helene and Toulme, supra.

The inhibitory nucleic acids can also be used to induce chemical inactivation or cleavage of the target genes or mRNA. Chemical inactivation can occur by the induction of crosslinks between the inhibitory nucleic acid and the target nucleic acid within the cell. Alternatively, irreversible photochemical reactions can be induced in the target nucleic acid by means of a photoactive group attached to the inhibitory nucleic acid. Other chemical modifications of the target nucleic acids induced by appropriately derivatized inhibitory nucleic acids may also be used.

Cleavage, and therefore inactivation, of the target nucleic acids can be affected by attaching to the inhibitory nucleic acid a substituent that can be activated to induce cleavage reactions. The substituent can be one that affects either chemical, photochemical or enzymatic cleavage. For example, one can contact an mRNA:antisense oligonucleotide hybrid with a nuclease which digests mRNA:DNA hybrids. Alternatively cleavage can be induced by the use of ribozymes or catalytic RNA. In this approach, the inhibitory nucleic acids would comprise either naturally occurring RNA (ribozymes) or synthetic nucleic acids with catalytic activity.

Inhibitory nucleic acids can also include RNA aptamers, which are short, synthetic oligonucleotide sequences that bind to proteins (see, e.g., L₁ et al., Nuc. Acids Res., 34:6416-24 (2006)). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

B. Inactivating Antibodies

Inhibition of signal transduction by sPLA2-IIA and integrin αvβ3 or α4β1 binding can be achieved with an inactivating antibody. An inactivating antibody can comprise an antibody or antibody fragment that specifically binds to any one of sPLA2-IIA and integrin chains αv, β3, α4, and β1 and subsequently abolishes or reduces the binding between sPLA2-IIA and integrin αvβ3 or α4β1. Inactivating antibody fragments include, e.g., Fab fragments, heavy or light chain variable regions, single complementary determining regions (CDRs), or combinations of CRDs with the desired target protein binding activity. An inactivating antibody for sPLA2-IIA-integrin binding can be a naturally occurring antibody derived from any appropriate organism, e.g., mouse, rat, rabbit, gibbon, goat, horse, sheep, etc., or an artificial antibody such as a single chain antibody (scFv), a chimeric antibody, or a humanized antibody.

The chimeric antibodies of the invention may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent chimeric antibody is based on an aggregation of chains.

C. Identification of sPLA2-IIA and Integrin Binding Inhibitors

One can identify compounds that are effective inhibitors of sPLA2-IIA and integrin αvβ3 or α4β1 binding by screening a variety of compounds and mixtures of compounds for their ability to suppress signal transduction via sPLA2-IIA and integrin via their interference of such binding. The testing can be performed in a cell-based system or in a cell-free system, using either the full length sequences of the sPLA2-IIA and integrin αvβ3 or α4β1 polypeptides or a minimal region or subsequence of at least one of sPLA2-IIA or integrin αvβ3 or α4β1 polypeptide that is sufficient to support the specific binding between sPLA2-IIA and the integrins.

One aspect of the present invention is directed to methods for screening compounds that have the activity to inhibit sPLA2-IIA specific binding with integrin αvβ3 or α4β1 and therefore to suppress a pro-inflammatory signal. Such compounds can be in the form of a mixture of suitable inhibitors, or each in substantially isolated form. An example of an in vitro binding assay can comprise an sPLA2-IIA polypeptide and integrin αvβ3 or α4β1 polypeptides (or fragments thereof responsible for sPLA2-integrin binding), where the level of sPLA2-IIA binding to integrin αvβ3 or α4β1 is determined in the presence or absence of a test compound. Optionally, one of the sPLA2-IIA or integrin polypeptides is immobilized to a solid substrate or support. A detectable label, e.g., a radioactive or fluorescent label, can be provided for sPLA2-IIA or integrin αvβ3 or α4β1, either directly or indirectly (through a second molecule that specifically recognizes sPLA2-IIA or one of the integrin chains αvβ3 and α4β1), to facilitate detection of sPLA2-IIA and integrin binding.

Another typical binding assay comprises cells expressing integrin αvβ3 or α4β1 on their surface and a free sPLA2-IIA polypeptide, where the level of sPLA2-IIA binding to integrin αvβ3 or α4β1 is determined in the presence or absence of a test compound. Suitable cells include any cultured cells such as mammalian, insect, microbial (e.g., bacterial, yeast, fungal), or plant cells. In some embodiments, the cells recombinantly express integrin αvβ3 or α4β1, whereas in other embodiments, the cells naturally express integrin αvβ3 or α4β1 on the cell surface. In this type of cell-based system, the level of sPLA2-IIA binding to integrin αvβ3 or α4β1 can be determined either directly by measuring the binding or indirectly by measuring the level of downstream effects of the binding such as activation of one or more mitogen-activated protein (MAP) kinases (e.g., extracellular signal-regulated kinases 1 or 2, ERK1 or ERK2), typically indicated by their increased phosphorylation at certain tyrosine residues. Increased proliferation rate in cells that express integrin αvβ3 or α4β1 upon exposure to sPLA2-IIA is also an indicator of sPLA2-IIA-integrin mediated signaling.

In some embodiments, the assays are designed to screen large chemical libraries by automating the assay steps and providing compounds from any convenient source to assays, which are typically run in parallel (e.g., in microtiter formats on microtiter plates in robotic assays).

In these screening assays it is optional to have positive controls to ensure that the components of the assays are performing properly. For example, a known inhibitor of sPLA2-IIA and integrin binding can be incubated with one sample of the assay, and the resulting change in signal determined according to the methods herein.

Essentially any chemical compound can be tested as a potential inhibitor of sPLA2-IIA and integrin binding by using methods of the present invention. Most preferred are generally compounds that can be dissolved in aqueous or organic (especially DMSO-based) solutions are used. It will be appreciated that there are many suppliers of chemical compounds, such as Sigma (St. Louis, Mo.), Aldrich (St. Louis, Mo.), Sigma-Aldrich (St. Louis, Mo.), and Fluka Chemika-Biochemica Analytika (Buchs, Switzerland).

Inhibitors of sPLA2-IIA and integin binding can be identified by screening a combinatorial library containing a large number of potential therapeutic compounds (potential modulator compounds). Such “combinatorial chemical libraries” can be screened in one or more assays, as described herein, to identify those library members (particular chemical species or subclasses) that display a desired characteristic activity. The compounds thus identified can serve as conventional “lead compounds” or can be directly used as potential or actual therapeutics.

Preparation and screening of combinatorial chemical libraries are well known to those of skill in the art. Such combinatorial chemical libraries include, but are not limited to, peptide libraries (see, e.g., U.S. Pat. No. 5,010,175; Furka, Int. J. Pept. Prot. Res., 37:487-493 (1991); and Houghton et al., Nature, 354:84-88 (1991)) and carbohydrate libraries (see, e.g., Liang et al., Science, 274:1520-1522 (1996); and U.S. Pat. No. 5,593,853). Other chemistries for generating chemical diversity libraries can also be used. Such chemistries include, but are not limited to: peptoids (PCT Pub. No. WO 91/19735); encoded peptides (PCT Pub. No. WO 93/20242); random bio-oligomers (PCT Pub. No. WO 92/00091); benzodiazepines (U.S. Pat. No. 5,288,514); diversomers, such as hydantoins, benzodiazepines and dipeptides (Hobbs et al., Proc. Nat. Acad. Sci. USA, 90:6909-6913 (1993)); vinylogous polypeptides (Hagihara et al., J. Amer. Chem. Soc., 114:6568 (1992)); nonpeptidal peptidomimetics with β-D-glucose scaffolding (Hirschmann et al., J. Amer. Chem. Soc., 114:9217-9218 (1992)); analogous organic syntheses of small compound libraries (Chen et al., J. Amer. Chem. Soc., 116:2661 (1994)); oligocarbamates (Cho et al., Science, 261:1303 (1993)); and/or peptidyl phosphonates (Campbell et al., J. Org. Chem., 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, Jan. 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); and benzodiazepines (U.S. Pat. No. 5,288,514)).

Alternatively, one can identify compounds that are suitable inhibitors of sPLA2-IIA and integrin specific binding by screening a variety of compounds and mixtures of compounds for their ability to suppress sPLA2-IIA or integrin αv, β3, α4, or β1 chain expression. Methods of detecting expression levels are well known in the art, and include both protein- and nucleic acid-based methods.

For example, a test agent can be contacted in vitro with cells expressing sPLA2-IIA. An agent that inhibits sPLA2-IIA expression is one that results in a decrease in the level of sPLA2-IIA polypeptide or transcript, as measured by any appropriate assay common in the art (e.g., Northern blot, RT-PCR, Western blot, or other hybridization or affinity assays), when compared to expression without the test agent. In some embodiments, a test nucleic acid inhibitor can be introduced into a cell, e.g., using standard transfection or transduction techniques, and the level of sPLA2-IIA expression detected. A typical decrease is a reduction in the expression level by at least 10%, or higher (e.g., at least 20%, 30%, 50%, 75%, 80%, or 90%) compared the level of expression in the absence of the test inhibitor.

### V. Inflammatory Responses and Conditions

Identification and diagnosis of inflammation, as well as methods of monitoring the effectiveness of a therapeutic regimen as described herein, are included in the present invention. As explained above, inflammation is generally characterized by redness, swelling, pain, and occasional loss of function. However, symptoms vary among tissues, so that some inflammatory conditions are not easily detectable (e.g., atherosclerosis).

Although the inflammatory response can play a role in the healing process by destroying, diluting, and isolating injurious agents and stimulating repair of the affected tissue, inflammatory responses can also be harmful. For example, inflammation results in leakage of plasma from the blood vessels. Although this leakage can have beneficial effects, it causes pain and when uncontrolled can lead to loss of function and death (such as adult respiratory distress syndrome). Anaphylactic shock, arthritis, and gout are among the conditions that are characterized by uncontrolled or inappropriate inflammation.

On a cellular level, an inflammatory response is typically initiated by endothelial cells producing molecules that attract and detain inflammatory cells (e.g., myeloid cells such as neutrophils, eosinophils, and basophils) at the site of injury or irritation. The inflammatory cells then are transported through the endothelial barrier into the surrounding tissue. The result is accumulation of inflammatory cells, in particular neutrophils. Such accumulation is easily detectable by one of skill.

Adaptive immune cells (T and B cells) are often involved in inflammatory conditions. These cells release cytokines and antibodies in response to the source of the irritation. Thus, an inflammatory response can also be detected by detecting a change in the level of inflammatory cytokines, e.g., in a localized region of irritation or in the serum or plasma of an individual. It will be appreciated by those of skill in the art that each of these symptoms can be detected in an individual for the purposes of diagnosis. Further, a subject undergoing therapy for an inflammatory condition can be monitored, for instance, by detecting any changes in severity of the symptoms. Such inflammatory conditions include rheumatoid arthritis, Alzheimer's disease, multiple sclerosis, and atherosclerosis.

### VI. Pharmaceutical Compositions and Administration

The present invention also provides pharmaceutical compositions comprising an effective amount of an inhibitor of sPLA2-IIA and integrin αvβ3 or α4β1 binding for inhibiting a pro-inflammatory signal, therefore useful in both prophylactic and therapeutic applications designed for various diseases and conditions involving undesired inflammation. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, intramuscular, intravenous, or intraperitoneal. The routes of administering the pharmaceutical compositions include systemic or local delivery to a subject suffering from a condition exacerbated by inflammation at daily doses of about 0.01-5000 mg, preferably 5-500 mg, of an inhibitor of sPLA2-IIA-integrin binding for a 70 kg adult human per day. The appropriate dose may be administered in a single daily dose or as divided doses presented at appropriate intervals, for example as two, three, four, or more subdoses per day.

For preparing pharmaceutical compositions containing an inhibitor of sPLA2-IIA-integrin binding, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., an inhibitor of sPLA2-IIA and integrin binding. In tablets, the active ingredient (the inhibitor) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of an sPLA2-IIA-integrin binding inhibitor with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., an inhibitor of sPLA2-IIA and integrin binding) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., an in activating sPLA2-IIA antibody) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing the inhibitor can be administered for prophylactic and/or therapeutic treatments. In therapeutic applications, compositions are administered to a patient already suffering from a condition that may be exacerbated by an undesirable inflammatory reaction in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the inhibitor per day for a 70 kg patient, with dosages of from about 5 mg to about 500 mg of the inhibitor per day for a 70 kg patient being more commonly used.

In prophylactic applications, pharmaceutical compositions containing an inhibitor of sPLA2-IIA-integrin binding are administered to a patient susceptible to or otherwise at risk of developing a disease or condition involving an undesirable inflammatory response in an amount sufficient to delay or prevent the onset of the symptoms. Such an amount is defined to be a “prophylactically effective dose.” In this use, the precise amounts of the inhibitor again depend on the patient's state of health and weight, but generally range from about 0.1 mg to about 2,000 mg of the inhibitor for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound sufficient to effectively inhibit the undesirable inflammatory response mediated by sPLA2-integrin binding in the patient, either therapeutically or prophylatically.

### VII. Therapeutic Applications Using Nucleic Acids

A variety of inflammatory conditions can be treated by therapeutic approaches that involve introducing an inhibitory nucleic acid into a cell such that the expression of sPLA2-IIA or integrin αvβ3 or α4β1 is suppressed in the cell. Those amenable to treatment by this approach include a broad spectrum of conditions involving undesirable inflammation. For discussions on the application of gene therapy towards the treatment of genetic as well as acquired diseases, see, Miller Nature 357:455-460 (1992); and Mulligan Science 260:926-932 (1993).

A. Vectors for Nucleic Acid Delivery

For delivery to a cell or organism, an inhibitory nucleic acid of the invention can be incorporated into a vector. Examples of vectors used for such purposes include expression plasmids capable of directing the expression of the nucleic acids in the target cell. In other instances, the vector is a viral vector system wherein the polynucleotide is incorporated into a viral genome that is capable of transfecting the target cell. In a preferred embodiment, the inhibitory nucleic acid can be operably linked to expression and control sequences that can direct transcription of sequence in the desired target host cells. Thus, one can achieve reduced expression of sPLA2-IIA or integrin αvβ3 or α4β1 under appropriate conditions in the target cell.

B. Gene Delivery Systems

As used herein, “gene delivery system” refers to any means for the delivery of an inhibitory nucleic acid of the invention to a target cell. Viral vector systems useful in the introduction and expression of an inhibitory nucleic acid include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the inhibitory nucleic acid is inserted into such vectors to allow packaging of the gene construct, typically with accompanying viral DNA, followed by infection of a sensitive host cell and expression of the gene of interest.

Similarly, viral envelopes used for packaging gene constructs that include the inhibitory nucleic acid can be modified by the addition of receptor ligands or antibodies specific for a receptor to permit receptor-mediated endocytosis into specific cells (see, e.g., WO 93/20221, WO 93/14188, and WO 94/06923).

Retroviral vectors may also be useful for introducing the inhibitory nucleic acid of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired inhibitory nucleic acid sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, the inhibitory nucleic acid, thus eliminating or reducing unwanted inflammatory conditions.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

C. Pharmaceutical Formulations

When used for pharmaceutical purposes, the inhibitory nucleic acid is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can further include a stabilizer, enhancer or other pharmaceutically acceptable carriers or vehicles. A pharmaceutically acceptable carrier can contain a physiologically acceptable compound that acts, for example, to stabilize the inhibitory nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

D. Administration of Formulations

The formulations containing an inhibitory nucleic acid can be delivered to any tissue or organ using any delivery method known to the ordinarily skilled artisan. In some embodiments of the invention, the nucleic acid is formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

The formulations containing the inhibitory nucleic acid are typically administered to a cell. The cell can be provided as part of a tissue or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the inhibitory nucleic acid is introduced into cells by such methods as microinjection, calcium phosphate precipitation, liposome fusion, ultrasound, electroporation, or biolistics. In further embodiments, the nucleic acid is taken up directly by the tissue of interest.

In some embodiments of the invention, the inhibitory nucleic acid is administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg inhibitory nucleic acid per patient are typical. Doses generally range between about 0.01 and about 50 mg per kilogram of body weight, preferably between about 0.1 and about 5 mg/kg of body weight or about 10⁸-10¹⁰ or 10¹² viral particles per injection. In general, the dose equivalent of a naked nucleic acid from a vector is from about 1 μg-100 μg for a typical 70 kg patient, and doses of vectors which include a retroviral particle are calculated to yield an equivalent amount of an inhibitory nucleic acid.

### VIII. Kits

The invention also provides kits for treating or preventing an inflammatory condition by inhibiting the specific binding between sPLA2-IIA and integrin αvβ3 or α4β1 according to the method of the present invention. The kits typically include a container that contains a pharmaceutical composition having an effective amount of an inhibitor for the specific binding between sPLA2-IIA and integrin αvβ3 or α4β1, as well as informational material containing instructions on how to dispense the pharmaceutical composition, including description of the type of patients who may be treated (e.g., a person suffering from or at risk of developing a condition involving undesired inflammatory response), the schedule (e.g., dose and frequency) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Experimental Procedures

Materials Recombinant soluble αvβ3 was synthesized as previously described (Takagi, J. et al., Nat. Struct. Biol., 8(5):412-416 (2001)). CHO cells expressing recombinant αvβ3 (designated β3-CHO cells) (Zhang, X. P. et al., J. Biol. Chem., 273(13):7345-7350 (1998)), wt or mutant α4 (Irie, A. et al., Embo J, 14(22):5550-5556 (1995)), and K562 human erythroleukemia cells that express human αv4 (Fleming, F. E. et al., Arch Virol, 152(6):1087-1101 (2007)) have been described. K562 cells that express human αvβ3 (αvβ3-K562) (Blystone, S. et al., Journal of Cell Biology., 127:1129-1137 (1994)) were provided by Eric Brown (UCSF). Human β3 was stably expressed in the CHO pgs745 mutant cells (Esko, J. D. et al., Proc. Natl. Acad. Sci. USA, 82(10):3197-3201 (1985)) deficient in xylosyltransferase, an essential enzyme in proteoglycan synthesis, as described (Zhang, X. P. et al., J. Biol. Chem., 273(13):7345-7350 (1998)).

Synthesis of sPLA2-IIA A cDNA fragment encoding sPLA2-IIA was amplified with sPLA2-IIA cDNA (ATCC) as a template using synthetic oligonucleotide primers 5′-GAAGATCTAATTTGGTG AATTTCCAC-3′ and 5′-GGAATTCTCAGCAACGAGGGGTGCTCCC-3′ by PCR. After digestion with Bgl II and Eco RI, the cDNA fragment was subcloned into the Bam HI/Eco RI sites of PET28a/amp vector. We generated the PET28a/amp vector by replacing the kanamycine-resistant gene of PET28a with the ampicilline-resistant gene of PET21a. We generated sPLA2-IIA as an insoluble protein in bacteria BL21 and purified it by Ni-NTA affinity chromatography under denatured conditions and refolded following the protocols (“Isolation of proteins from inclusion bodies” in www.its.caltech.edu/˜bjorker/protocols/). Briefly, purified proteins in 8 M urea were diluted into refolding buffer (100 mM Tris-HCl, pH 8.0, 400 mM L-Arg, 2 mM EDTA, 0.5 mM oxidized glutathione, 5 mM reduced glutathione, and protease inhibitors) and kept for 8 h at 4° C., and then concentrated by ultrafiltration. The refolded sPLA2-IIA was more than 90% homogeneous in SDS-PAGE. We performed site-directed mutagenesis by QuickChange method (Wang, W. and Malcolm, B. A., BioTechniques, 26:680-682 (1999)). The presence of mutations was confirmed by DNA sequencing. To remove endotoxin, we washed the Ni-NTA resin with 1% Triton X-114 before eluting the bound protein. We confirmed that the sPLA2-IIA (wt and mutants) had no detectable endotoxin as tested by the Limulus amebocyte lysate assay (Fisher Scientific, Fair Lawn, N.J.).

Binding of soluble αvβ3 to immobilized sPLA2-IIA sPLA2-IIA was immobilized to wells of 96 well microtiter plates and the remaining protein-binding sites were blocked by BSA as described (Mori, S. et al., J Biol Chem, 283:18066-18075 (2008)). Soluble recombinant αvβ3 at 5 μg/ml in Hepes-Tyrodes buffer supplemented with 1 mM MnCl₂ was added to the well and incubated for 2 h. Bound αvβ3 was measured using anti-integrin β3 (mAb AV-10) followed by HRP-conjugated goat anti-mouse IgG and peroxidase substrates.

Binding of FITC-labeled sPLA2-IIA to integrins on the cell surface sPLA2-IIA was labeled with FITC using Fluorescein labeling kit (Pierce) according to the manufacturer's instructions. Cells were harvested with 3.5 mM EDTA in PBS. Cells were double-labeled with a) FITC-labeled sPLA2-IIA (10 μg/ml in the presence of 10 mM Mg²⁺ at room temperature for 30 min), and b) with non-blocking anti-human integrin β3 subunit mAb AV-10 and PE (phycoerythrin) conjugated secondary antibody. Bound FITC (FL1) and PE (FL2) were quantified in flow cytometry.

Surface plasmon resonance study Recombinant soluble integrin αvβ3 was immobilized to Biacore Sensor chip CM5 (Biacore, Piscataway, N.J.) by the standard amine coupling method. Two-fold serial diluted sPLA2-IIA and its mutants R74E/R100E (ranging from 2 nM to 500 pM) and H47Q (ranging from 4 nM to 1 nM) in running buffer HBS-P buffer containing 1 mM of Mn²⁺ were injected for 3 min at the flow rate of 50 μL/min. Then the sensor chip was washed with the running buffer alone at the same flow rate for another 5 min (the dissociation phase). Two consecutive one-minute injections of 0.5 M, pH 8 EDTA solution at the same flow rate were used to regenerate the chip for another cycle of injection. The resonance unit elicited from the reference flow cell was subtracted from the resonance unit elicited from the integrin flow cell to eliminate the non-specific protein-flow cell interaction and the bulk refractive index effect. The recorded binding curves were fitted with the “1:1 binding with drifting baseline model” by using the Biaevaluation Version 4.1.

Cell proliferation and MAP kinase activation K562 cells and human monocytic lymphoma U937 cells were maintained in RPM11640 medium supplemented with 10% FCS. Cells were plated in 96-well plates (1×10⁴ cells/well), and serum-starved for 48 h at 37° C. in 5% CO₂ atmosphere. Cells were then treated with or without sPLA2-IIA in medium without serum for 48 h. Cell proliferation was determined by MTS assays using the Aqueous Cell Proliferation Assay Kit (Promega). For MAP kinase activation assays, cells were serum starved in RPMI1640 medium supplemented with 0.4% FCS for 24 h, and stimulated with Wt and mutant sPLA2-IIA (0.5 μg/ml) for 10 min at 37° C. ERK1/2 activation was measured as described (Mori, S. et al., J Biol Chem, in press (2008)).

Other methods We performed docking simulation of interaction between sPLA2-IIA and integrin αvβ3 using the AutoDock3 as previously described (Mori, S. et al., J Biol Chem, in press (2008)). Adhesion assays were performed as described previously (Eto, K. et al., J. Biol. Chem., 277:17804-17810 (2002)). mAb 7E3 was used at 10 μg/ml. We assayed PLA2 activity by arachidonoyl-Thio-PC hydrolysis (Cayman Chemicals, Ann Arbor, Mich.) as described (Reynolds, L. J. et al., Anal. Biochem., 217:25-32 (1994)).

### Results

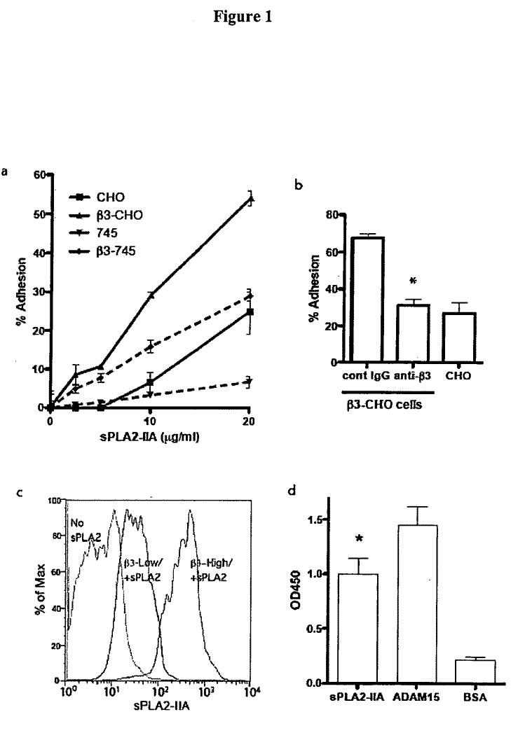

sPLA2-IIA binds to integrin αvβ3 To test if sPLA2-IIA binds to integrin αvβ3, we used CHO cells and the proteoglycans-deficient variants of CHO cells (pgs745) (Esko, J. D. et al., Proc. Natl. Acad. Sci. USA, 82(10):3197-3201 (1985)) expressing recombinant αvβ3 (designated β3-CHO and β3-745 cells, respectively). We found that f3-CHO and 03-745 cells adhered to immobilized sPLA2-IIA at a much higher level than mock-transfected CHO or 745 cells (FIG. 1a). Consistent with the previous report that proteoglycans support binding of positively charged sPLA2-IIA to the cell surface (Fuentes, L. et al., FEBS Lett, 531(1):7-11 (2002)), mock-CHO cells adhered to sPLA2-IIA better than mock-transfected 745 cells. These results suggest that the difference in adhesion between 03-CHO and CHO or between 03-745 and 745 reflects the integrin αvβ3-mediated adhesion to sPLA2-IIA. We found that mAb against human integrin (33 subunit (mAb 7E3) effectively reduced the adhesion of (3-CHO cells to the background level (from 67% to about 30%) (FIG. 1b), indicating that the adhesion is specific to αvβ3. These results indicate that αvβ3 mediated cell adhesion to sPLA2-IIA, that proteoglycans partly supported cell adhesion to sPLA2-IIA.

Next we tested if soluble sPLA2-IIA binds to cell surface αvβ3. We found that FITC-sPLA2-IIA bound at much higher levels to cells expressing high-level αvβ3 (β3-high) than to cells expressing little αvβ3 (β3-low) (FIG. 1c), indicating the significant contribution of αvβ3 in sPLA2-IIA binding. The low-level binding of sPLA2-IIA to αvβ3-low cells probably represents contribution of proteoglycans and other receptors.

We next demonstrated that recombinant soluble αvβ3 bound to immobilized sPLA2-IIA in ELISA-type assays (FIG. 1d). Soluble αvβ3 bound to the disintegrin domain of ADAM15, a known αvβ3 ligand (Zhang, X. P. et al., J. Biol. Chem., 273(13):7345-7350 (1998)) (as a positive control), but did not significantly bound to BSA (as a negative control). These results indicate that αvβ3 directly binds to sPLA2-IIA. We showed that soluble sPLA2-IIA bound to immobilized αvβ3 in surface plasmon resonance studies at a high affinity (see below).

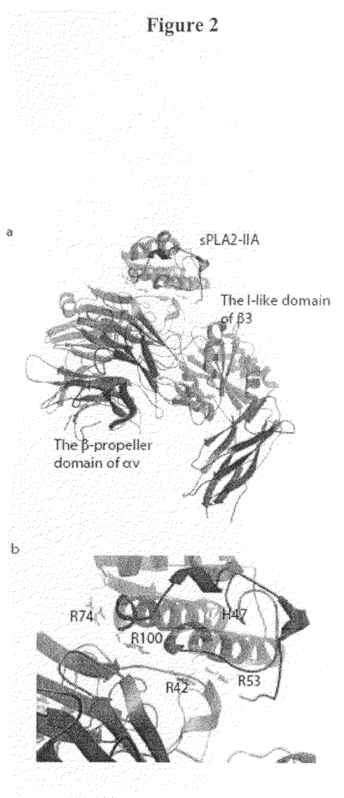

Docking simulation of interaction between integrin and sPLA2-IIA To determine how sPLA2-IIA binds to integrin αvβ3, we performed docking simulation by using AutoDock3. AutoDock is a set of docking tools widely used for predicting the conformation of small ligands bound to receptors (Goodsell, D. S. and Olson, A. J., Proteins, 8(3):195-202 (1990); Morris, G. M. et al., J. Comp. Chem., 19:1639-1662 (1998); Morris, G. M. et al., J. Comput. Aided Mol. Des., 10(4):293-304 (1996)), and the methods has been used to predict protein-protein complex poses (Saphire, E. O. et al., Science, 293(5532):1155-1159 (2001)). We performed 50 dockings, each one starting with a random initial position and orientation of sPLA2-IIA (PDB code 1DCY1 and 1AYP) with respect to the headpiece of αvβ3 (PDB code 1L5G). The results were clustered together by positional RMSD (0.5 Angstrom) into families of similar poses. Twenty-four of the 50 docking poses clustered well with the lowest docking energy (cluster 1), with a docking energy −26.1 Kcal/mol with 1DCY1 and −25.5 Kcal/mol with 1AYP. These results predict that the docking pose of cluster 1 represents the most probable stable sPLA2-IIA pose upon binding to αvβ3 (FIG. 2a). While the poses obtained by using two structures are slightly different, the integrin-binding sites are overlapping. This model predicts that the integrin-binding interface of sPLA2-IIA with integrin αvβ3 does not include the catalytic center of sPLA2-IIA (e.g., His-47). The interface on the αvβ3 side contains several αv (green) or 03 (red) residues that have been shown to be critical for ligand binding by mutagenesis and crystallographic studies (Takagi, J. et al., J. Biochem, 121:914-921 (1997); Humphries, J. D. et al., J. Biol. Chem., 275(27):20337-20345 (2000); Xiong, J. P. et al., Science, 296(5565):151-155 (2002)). Thus the predicted docking model is consistent with the previous biochemical studies of integrin-ligand interaction.

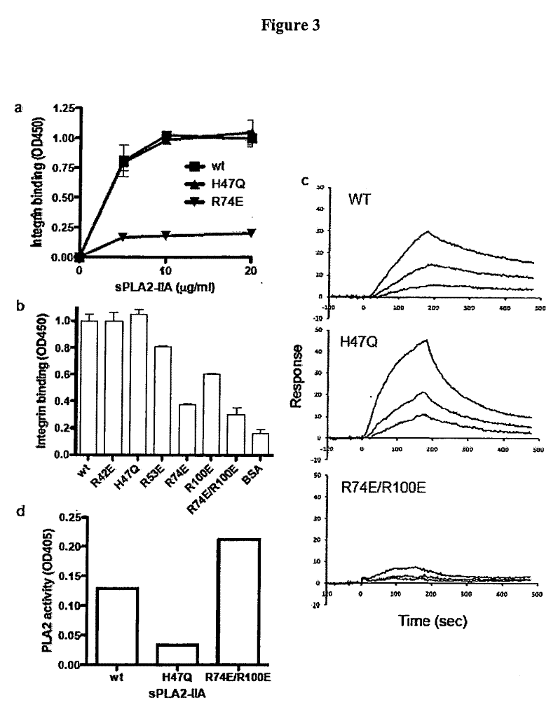

Mutagenesis study of the predicted integrin-binding interface of sPLA2-IIA To test if the docking model is correct, we introduced several mutations within the predicted interface of sPLA2-IIA with integrin αvβ3. Positively charged amino acids at the predicted interface common to 1AYP and 1DCY (Arg-42, Arg-53, Arg-74, and Arg-100) were mutated to Glu (charge reversal mutagenesis) (FIG. 2b). We found that the R74E and R100E mutations in sPLA2-IIA reduced the binding to soluble αvβ3, while the R42E and R53E mutations had little or no effect on integrin binding (FIGS. 3a and 3b). We generated the catalytically inactive mutant of sPLA2-IIA by mutating His-47 to Gln (the H47Q mutation) as a control. The H47Q mutation did not affect the binding to soluble αvβ3. These results are consistent with the prediction by docking simulation. The combined R74E/R100E mutation effectively reduced the binding of sPLA2-IIA to soluble αvβ3 (FIG. 3b) and was used for further analysis of the role of integrins in sPLA2-IIA signaling.

We determined binding kinetics of wt and mutant sPLA2-IIA to soluble αvβ3 using surface plasmon resonance (SPR) (FIG. 3c). Wt and H47Q sPLA2-IIA showed high affinity to αvβ3 (KD=2.11×10⁻⁷M and 4.47×10⁻⁸M, respectively) and the R74E/R100E mutant showed much lower affinity (KD=1.08×10⁻⁶M). This is consistent with the results obtained by ELISA-type binding assays.

PLA2 activity was measured to confirm that the integrin-binding-defective mutation did not affect catalytic activity (FIG. 3d). The data suggest that the H47Q mutation reduced PLA2 activity (while its integrin binding was not affected). In contrast, the R74E/R100E mutation did not affect PLA2 activity (while its integrin binding was suppressed).

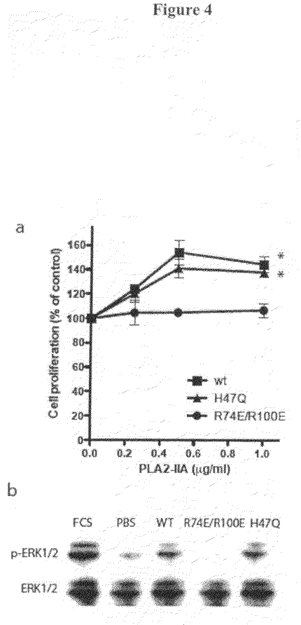

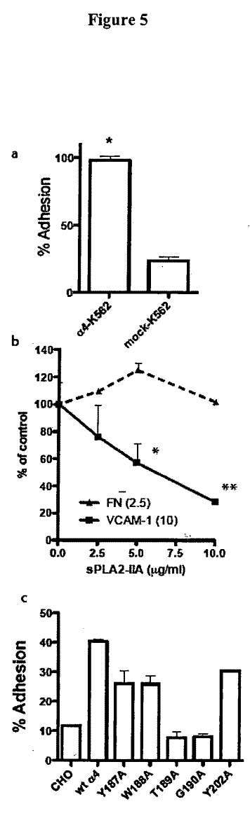

sPLA2-IIA-induced proliferation of monocytic cells in an integrin-dependent manner—It has been reported that sPLA2-IIA-induced proliferation of LNCap prostate cancer cells in a dose-dependent manner (Sved, P. et al., Cancer Res, 64(19):6934-6940 (2004)) and induced resistance to apoptosis in baby hamster kidney (BHK) cells (Zhang, Y. et al., J Biol Chem, 274(39):27726-27733 (1999)). We tested if the integrin-binding-defective or catalytically inactive mutations affect sPLA2-IIA's ability to induce proliferative signals. Notably, we found that wt sPLA2-IIA and H47Q-induced robust proliferation of monocytic U937 cells, but R74E/R100E did not (FIG. 4a). These results suggest that integrin binding to sPLA2-IIA plays a critical role in sPLA2-IIA-induced cell proliferation, but catalytic activity is not important in this process. Consistent with this observation, wt sPLA2-IIA and H47Q-induced, but R74E/R100E did not induce, ERK1/2 activation in U937 cells (FIG. 4b). While it has been reported that U937 cells express αvβ3 (Nath, D. et al. J. Cell Sci., 112(Pt 4):579-587 (1999)), 7E3 did not block adhesion of U937 cells to sPLA2-IIA (not shown), suggesting that other receptors are involved in the binding of sPLA2 to U937 cells. We hypothesized that integrin α4β1, a major integrin in U937 cells (Hemler, M. E. et al., J. Biol. Chem., 262(24):11478-11485 (1987)), may be involved in sPLA2-IIA signaling in U937 cells. We tested this hypothesis using K562 cells that express recombinant α4β1 (α4-K562 cells). α4-K562 cell adhered to immobilized wt sPLA2-IIA better than mock-transfected K562 cells (FIG. 5a), suggesting that α4β1 interacts with sPLA2-IIA. However, a small molecular weight α4β1 ligand (LLP2A) (Peng, L. et al., Nat Chem Biol, 2(7):381-389 (2006)) or anti-α4β1 mAb SG73 or P4C2 (Irie, A. et al., Embo J, 14(22):5550-5556 (1995)) did not block α4β1 binding to sPLA2-IIA in transfected K562 cells (data not shown). To confirm that sPLA2-IIA binds to α4β1, we tested if sPLA2-IIA competes with known α4β1-specific ligand such as vascular cell adhesion molecule (VCAM)-1 for binding to α4β1. We found that sPLA2-IIA suppressed adhesion of U937 cells to VCAM-1 (FIG. 5b), whereas sPLA2-IIA did not suppress cell adhesion to α4β1-specific ligand fibronectin domains 8-11. K562 cells have endogenous α5β1. This suggests that sPLA2-IIA competed with VCAM-1 for binding to α4β1.

To confirm that sPLA2-IIA binds to α4β1, we mapped the sPLA2-IIA binding site in the α4 subunit. We previously identified several amino acid residues in the α4 subunit (e.g., Tyr-187 and Gly-190) that are critical for VCAM-1 and CS-1 binding (Irie, A. et al., Embo J, 14(22):5550-5556 (1995)) and for binding of LLP2A (Peng, L. et al., Nat Chem Biol, 2(7):381-389 (2006)) to α4 by introducing point mutations in the α4 subunit. We tested if these α4 mutations affect sPLA2-IIA binding to α4β1. We found that mutating Tyr-189 and Gly-190 of α4 to Ala blocked binding to sPLA2-IIA (FIG. 5c), suggesting that sPLA2-IIA binding site in α4 is close to or overlaps with the VCMA-1 or CS-1 binding sites. We obtained similar results using K562 cells that express the α4 mutants (data not shown). These findings are consistent with the observation that sPLA2-IIA and VCAM-1 competed for binding to α4β1.

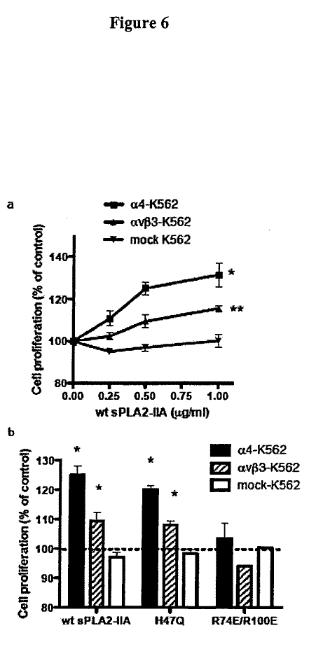

To test if αvβ3 and α4β1 individually mediate sPLA2-IIA-induced cell proliferation, we used K562 cells that over-expressed αvβ3 or α4β1 (αvβ3- and α4-K562 cells, respectively). sPLA2-IIA-induced proliferation of α4-K562 cells, and to a less extent, of αvβ3-K562 cells. sPLA2-IIA did not induce proliferation of mock-transfected K562 cells (FIG. 6a). Consistent with the results with U937 cells, H47Q-induced proliferation of αvβ3- and α4-K562 cells, but R74E/R100E did not induce proliferation of αvβ3- or α4-K562 (FIG. 6b). These results suggest that sPLA2-IIA-induced proliferation of K562 cells required the binding of sPLA2-IIA to α4β1 or αvβ3, but did not require catalytic activity of sPLA2-IIA.

### Discussion

The present study establishes for the first time that human sPLA2-IIA specifically bound to integrin αvβ3 at a high affinity (KD 2×10⁻⁷M). Using docking simulation and mutagenesis, we developed an integrin-binding-defective mutation of sPLA2-IIA (the R74E/R100E mutation) that effectively reduced αvβ3 binding without affecting catalytic activity. In contrast the H47Q mutation destroyed catalytic activity, but did not reduce αvβ3 binding. SPR studies showed that the R74E/R100E mutation markedly reduced the binding affinity to αvβ3, but the H47Q mutant did not. These results are consistent with the prediction from the simulation, and that the integrin-binding site is distinct from the catalytic center or the M-type receptor-binding site, in which Gly-30 and Asp-49 of sPLA2-IIA are involved (Lambeau, G. et al., J Biol Chem, 270(10):5534-5540 (1995)).

Integrin αvβ3 is a ubiquitous receptor that is expressed on a variety of cell types (Eliceiri, B. P. and Cheresh, D. A., J. Clin. Invest., 103(9):1227-1230 (1999); Byzova, T. V. et al., Thromb. Haemost., 80(5):726-73444,45 (1998)). Consistent with its expression profile in vivo, αvβ3 plays a key role in the initiation or progression of several human diseases, including rheumatoid arthritis, cancer, and ocular diseases, and cardiovascular diseases (Eliceiri, B. P. and Cheresh, D. A., J. Clin. Invest., 103(9):1227-1230 (1999); Byzova, T. V. et al., Thromb. Haemost., 80(5):726-73444,45 (1998)). Endothelial cells are primary targets in angiogenesis in chronic inflammation and cancer, and activated endothelial cells express high levels of αvβ3 (Eliceiri, B. P. and Cheresh, D. A., J. Clin. Invest., 103(9):1227-1230 (1999)). Macrophages represent a major mononuclear cell population in inflammation (Antonov, A. S. et al., Am. J. Pathol., 165(1):247-258 (2004)), and macrophages express high-level αvβ3. Its expression is modulated by several cytokines (e.g., interleukin-4, tumor necrosis factor-α) and growth factors (e.g., platelet-derived growth factor, fibroblast growth factor). αvβ3 is consistently detected on the macrophages in early and advanced human atherosclerotic lesions, and its expression is up regulated by atherogenic stimuli (oxidized low-density lipoprotein, macrophage colony-stimulating factor) in vitro (Antonov, A. S. et al., Am. J. Pathol., 165(1):247-258 (2004)). These reports suggest that sPLA2-IIA and αvβ3 co-exist in the inflammatory lesion and directly connect the pro-inflammatory action of sPLA2-IIA and αvβ3, the newly identified receptor of sPLA2-IIA.

We also presented evidence that α4β1 that is widely expressed in immune-competent cells (Hynes, R. O. et al., Cell, 110(6):673-687 (2002)) mediated sPLA2-IIA binding using K562 cells that express recombinant α4. Although mAbs or small-molecular weight antagonist tested against α4 did not significantly inhibit α4β1-sPLA2-IIA interaction, we showed that sPLA2-IIA competed with VCAM-1 for binding to α4β1. Also, amino acid residues of α4 (Tyr-189 and Gly-190) that are critical, or close to the critical, residues for VCAM-1 and CS-1 binding were also critical for sPLA2-IIA binding. These findings suggest that sPLA2-IIA binds to α4β1 in a ligand-binding site common to those for other known α4β1 ligands.

We showed that wt sPLA2-IIA and H47Q-induced proliferation and ERK1/2 activation in U937 cells (αvβ3+, α4β1+), while R74E/R100E did not, suggesting that sPLA2-IIA-induced proliferative signals of monocytic cells in an integrin-dependent manner. These observations directly connect the pro-inflammatory functions of sPLA2-IIA and integrins. Although relative contribution of α4β1 and αvβ3 in sPLA2-IIA-induced proliferative signals in U937 cells is unclear, we showed α4β1 and to a less extent αvβ3 can individually mediate cell proliferation using αvβ3- and α4-K562 cells. In both cases sPLA2-IIA-induced cell proliferation in an integrin-dependent and catalytic activity-independent manner. Because K562 cells have very low proteoglycans (Zhang, H. C. et al., J. Med. Chem., 44(7):1021-1024 (2001)), the effect of sPLA2 binding to proteoglycans is not important in this cell type.

It has been reported that specific inhibitors of sPLA2-IIA catalytic activity S-5920/LY315920Na and S-3013/LY333013 failed to demonstrate a significant therapeutic effect in rheumatoid arthritis (Bradley, J. D. et al., J Rheumatol, 32(3):417-423 (2005)) and asthma (Bowton, D. L. et al., J Asthma, 42(1):65-71 (2005)). The present results suggest that sPLA2-IIA-integrin interaction is a novel potential therapeutic target in inflammation. It would be important to develop antagonists that effectively block this interaction to fully evaluate the significance of this interaction in future studies.

All patents, patent applications, and other publications cited in this application, including published amino acid or polynucleotide sequences, are incorporated by reference in the entirety for all purposes.

What is claimed is:

1. A method for identifying an inhibitor for integrin-sPLA2-IIA binding, comprising the steps of:
(a) contacting a test compound with sPLA2-IIA and integrin αvβ3 or integrin α4β1, under conditions that permit specific binding between sPLA2-IIA and integrin αvβ3 or integrin α4β1;
(b) determining the level of specific binding between sPLA2-IIA and integrin αvβ3 or integrin α4β1, wherein a decrease in the level of specific binding compared to a control level of specific binding between sPLA2-IIA and integrin αvβ3 or integrin α4β1 under the same conditions but in the absence of the test compound indicates the compound as an inhibitor for integrin-sPLA2-IIA binding.

2. The method of claim 1, wherein integrin αvβ3 or integrin α4β1 is present on the surface of a cell.

3. The method of claim 2, wherein integrin αvβ3 or integrin α4β1 is recombinantly expressed.

4. The method of claim 1, wherein sPLA2-IIA is immobilized on a solid support.

5. The method of claim 1, wherein integrin αvβ3 or α4β1 is immobilized on a solid support.

6. The method of claim 1, wherein sPLA2-IIA is labeled with a fluorescent dye.

7. The method of claim 6, wherein the fluorescent dye is fluorescein isothiocyanate (FITC).

8. The method of claim 2, wherein the level of specific binding between sPLA2-IIA and integrin is determined by measuring the level of activation of at least one MAP kinase.

9. The method of claim 8, wherein the MAP kinase is ERK1 or ERK2.

10. The method of claim 2, wherein the level of specific binding between sPLA2-IIA and integrin is determined by measuring the level of proliferation of the cell.

11. The method of claim 10, wherein the cell is U937 human monocytic lymphoma cell.

12. The method of claim 10, wherein the cell is K562 cell.

13. A method for treating or preventing an inflammatory condition, comprising the step of administering to a subject an effective amount of an inhibitor for sPLA2-IIA and integrin αvβ3 binding or sPLA2-IIA and integrin α4β1 binding.

14. The method of claim 1, wherein the inhibitor is an inactivating antibody of sPLA2-IIA or integrin αv, α4, β1, or β3.

15. The method of claim 1, wherein the inhibitor is an inhibitory nucleic acid comprising a sequence complementary to an sPLA2-IIA or integrin αv, α4, β1, or β3 polynucleotide.

16. A composition comprising (1) an effective amount of an inhibitor for sPLA2-IIA and integrin αvβ3 binding or sPLA2-IIA and integrin α4β1 binding and (2) a pharmaceutically acceptable carrier.

17. The composition of claim 16, wherein the inhibitor is an inactivating antibody of sPLA2-IIA or integrin αv, α4, β1, or β3.

18. The composition of claim 16, wherein the inhibitor is an inhibitory nucleic acid comprising a sequence complementary to an sPLA2-IIA or integrin αv, α4, β1, or β3 polynucleotide.

19. The composition of claim 16, further comprising an additional therapeutic compound.

20. A kit for treating an inflammatory condition, said kit comprising the composition of claim 16.
